(19) United States
(12) Patent Application Publication
(10) Pub. No.: US 2016/0262912 A1
(43) Pub. Date: Sep. 15, 2016

(54) Temporary, modular, hip joint with neck-length modification mechanism

(71) Applicants: Brian G. Burnikel, Greer, SC (US); Alexander J. Burnikel, Greer, SC (US)

(72) Inventors: Brian G. Burnikel, Greer, SC (US); Alexander J. Burnikel, Greer, SC (US)

(21) Appl. No.: 14/544,999

(22) Filed: Mar. 13, 2015

(51) Int. Cl. A61F 2/46; F16H 19/04; A61F 2/36

(52) U.S. Cl. CPC A61F 2/4657; A61F 2/3609; A61F 2/4684; F16H 19/04; A61F 2002/3645; A61F 2002/4658; A61F 2220/0025; A61F 2310/00017; A61F 2310/00023

(57) ABSTRACT

A temporary neck for a hip joint prosthesis comprising a mechanism that will allow the length of the temporary neck to be varied while in place, during implant surgery and after the stem and base of a permanent prosthesis have been positioned. In one best mode the variation in length is driven by rotating a pinion gear that engages a rack gear connected to the interior of the side walls of a moveable unit, and the ball is connected by a stud to the front wall of the moveable unit; thus the ball moves the same distance as the moveable unit, and that distance is a direct function of gear tooth spacing and number of rotations of the gear. In an alternative mechanism, a threaded drive axle (worm gear) moves in response rotation an adjustment base (wheel gear) in which the base comprises a drive disk with a female, threaded bore. The axle is secured to the pressure disk at one end and free at the other. The position of the drive disk (hence bore) is static; thus as the drive disk rotates and the threads are engaged, the pressure disk moves and the ball moves with it. Both devices include visual references to determine precisely the distance that has been traveled (generally in mm).

### PRIORITY

This Application claims priority of U.S. Provisional Application No. 61/967,315 filed Mar. 17, 2014 and which Provisional Application is hereby incorporated in its entirety.

### FIELD OF THE INVENTION

The claimed invention is generally related to orthopaedic surgery. More specifically, it is related to fitting prosthetic hip joints, and more specifically it is a modification of the neck segment of a temporary, prosthetic hip joint and related method to determine the optimum length of the neck of the permanent joint by use of a temporary prosthesis with an extendable/retractable neck determine the optimum length of the neck of the permanent prosthesis.

### INTRODUCTION

Growth of Medical Speciality and Commercial Growth

The current, basic hip joint prosthesis traces to the work of Sir John Charnley, British orthopaedic surgeon working at Wrightington Hospital. By the 1970's, Charnley's design effectively replaced all of the prosthetic designs in use; earlier designs common until the 1960's followed the work of Burmese physician Dr. San Baw, who used ivory implants. Charnley's design comprised three elements: a one piece stainless steel femoral stem and head element, a plastic (polyethylene), and a cement.

The growth of joint replacement surgery is reflected in several facts: the sheer increase in the number of replacements performed annually over the past decade. The success of increasingly complex procedures and the number of physicians limiting their practices to joint replacement surgery and closely related surgery. In addition. The growth is reflected in the in the increased number of manufacturers of prosthetic devices related to joint replacements, in addition to specific joints. By way of example, not endorsement of those named or criticism of any omitted, major national/international suppliers include De Puy Synthesis (a part of Johnson and Johnson), Smith and Nephew, Inc. Biomet, Inc., Exacter, Inc., and Wright Medical Technology, all US business entities and all involved in varying degrees in research and development of specific devices, as well as manufacturing and marketing. A very limited internet search reveals over 100 manufacturers/suppliers of hip joints.

Technical Growth Reflected in Number and Diversity of Issued Patents, Prior Art.

An early exoprosthetic device addresses treating some fractures as well as joint replacement with an external, non-implant, implant device. U.S. Pat. No. 2,251,209 issued Jul. 29, 1941 to Stader addresses two orthopaedic issues with respect to fractures: recognizing that the fracture must be immobilizes and that frequently immobilization involved a joint. The bone splint disclosed in the '209 patent provides the necessary immobilization of the fracture site while allowing movement of the joint, thereby reducing discomfort and muscular deterioration as a result of prolonged immobilization.

U.S. Pat. No. 3,102,536 issued Sep. 9, 1963 to Rose and Wright clearly represents the technology of the Charnley hip prosthesis. The patent describes and claims a neck modification of a hip prothesis that allowed interchanging the ball to achieve, at that time, a more nearly optimum fit/placement of the ball and cup.

Contact of the surface of the prosthetic ball with the surface of the cup presents several continuing challenges: the need for some type or form of lubrication to ensure smooth contact between the surfaces and some means to distribute the hip load on the surface of the hip ball uniformly to the surface of the cup, rather than having undesirable pressure points from the position of the ball in the cup during actual use. Yakich in U.S. Pat. No. 3,864,758 issued Feb. 11, 1975 disclosed the use of a double wall of a spherical ball filled with a lubricating fluid and disposed in the space between the surfaces of the ball and cup as an effective way to distribute load (pressure) uniformly on the cup and to provide a lubrication effect along the ball/cup interface. The prosthesis further allows adjustment in the length and rotation bearing to achieve optimum positioning of the ball/cup.

The extended recovery from total hip replacement surgery (as well as other, comparable procedures) has been greatly reduced as a result of implantations employing new technologies in minimal invasive surgery as well as advances in recommended preoperative physical therapy and post-operative care and therapy. vonRecum in U.S. Pat. No. 4,488,319 issued Dec. 18, 1984 disclosed and claimed a two step hip replacement surgical procedure that reduced total recovery time and enhanced bony interfacial fixation of the stem implant. Briefly, the procedure involved implanting the stem with ball attached in the first procedure, allowing the natural socket (cup) to remain. The stem was treated to encourage bony fixation, accelerated in part from “use” prior to the second procedure in which the natural socket was replaced. A two piece hip prosthesis is also described and claimed. This procedure currently, apparently in wide spread use, in light of the evolution of minimally invasive surgical technologies and devices, reflects the use and evolution of modular prosthetic devices.

U.S. Pat. No. 581,928 issued Jan. 26, 1993 to Bolesky, Smith, and Whitcraft discloses and claims a modular hip prosthesis for partial hip replacement comprising three major elements. The prosthesis is distributed in kits such that various combinations of sizes of the elements may be assembled.

U.S. Pat. No. 6,319,286, issued Nov. 20, 2001 to Fernandez, Miller, and Mauldin describes a variation of a modular hip prosthesis. The prosthesis of the '286 patent comprises three major elements: a proximal segment that includes a neck that is lockingly engageable with a femoral head component and a male, tapered portion; a distal segment that includes a proximal end and a distal tip—the distal segment is further formed with a male tapered portion adjacent to its proximal end; and a metaphyseal segment with a proximal end and a distal end. A first, female tapered portion of the metaphyseal lockingly engages the male portion of the male, tapered portion of the proximal segment, and a second, female portion lockingly engages the male portion of the distal segment. The three modular components are interchangeable, there by affording a near optimal fit of the prosthetic joint.

U.S. Pat. No. 7,022,141 issued Apr. 4, 2006 to K. Dwyer, D. Daniels, and B. Parker provides an instrument to replicate or measure the angular orientation of a prosthesis to a second component. The instrument allows the surgeon to find and use as a reference point relations to a “landmarks” such as anteriorly bowed intra-medullary canal, a stable feature, even for revision surgeries in which other “land marks” may be destroyed.

U.S. Pat. No. 7,794,503 issued to D. Daniels, K. Dwyer, and D. Mattingly on Sep. 10, 2010 describes and claims a “trialing” system and method for modular hip joint replacement. The disclosed technology allows evaluation and replication of the anatomic anteversion rotational angle of the femur. The prosthetic stem is positioned within the femur. A proximal trial body assembly is mounted on the proximal portion of the distal stem component to allow rotation of a trial neck component which can be adjusted during surgery to determine final positioning of the permanent neck.

In one further example, U.S. Pat. No. 7,854,737, issued to D. Daniels, K. Dwyer, and D. Mattingly on Dec. 21, 2010, describes and claims an instrument and method for trialing for a modular hip stem. (See U.S. Pat. No. 7,022,141 for background and related technology and claims). A trial component fits into the cavity of the long bone (femur) and provides assistance in a trial reducting associated with joint arthroplasty surgery. The trial component comprises a stem portion and a neck portion fixedly connected to the stem portion in a plurality of selectable positions with respect to the stem, allowing the selection of the optimum position for the permanent positioning.

Goals and Objectives

A first goal and objective of the invention is a temporary, adjustable, modular hip joint prosthesis in which the overall length of the neck may be increased or decreased by a temporary neck element after the stem has been positioned in the femur and connected to the body of the prosthesis and the neck has been connected to the body and the ball.

A second goal and objective of the invention is a temporary, adjustable, modular hip joint prosthesis in which a slide unit positioned in a housing cavity of the adjustable base portion of a temporary neck element device wherein the slide unit moves forward and backwards in response to operation of a travel guide gear system.

A third goal and objective of the invention is a temporary, adjustable, modular hip joint prosthesis in which the ball is removably connected to the front wall of the slide unit and moves with the slide unit in response to operating the travel guide gear system.

The fourth goal and objective of the invention is a temporary, adjustable, modular hip joint prosthesis in which operating the guide gear system changes the length of the neck in small increments and the magnitude of the cumulative changes are visible on a fixed scale on stationary and corresponding moving parts of the gear system so length changes can be followed.

A fifth goal an objective of the invention is a temporary, adjustable, modular hip joint prosthesis in which the adjustable base, housing cavity, slide unit, and guide gear system (the temporary neck element) are replaced with a threaded screw system by which the temporary neck can be lengthened or shortened.

These and other related goals and objectives can be achieved by an invention described in the following Brief Description of the Invention.

### BRIEF DESCRIPTION OF THE INVENTION

The functional heart of the temporary, adjustable, modular hip joint prosthesis is a neck element that comprises a mechanism that allows the neck length to be changed after the stem has been implanted in the long bone (femur) of the leg and has also been connected to the base element of the prosthetic device; for one best mode by which the neck may be lengthened, the temporary element has an adjustment base and a sliding unit; the adjustment base comprises a chamber with a back wall and side walls; the adjustment unit is secured to the prosthesis base by a threaded stud, the stem is anchored in the femur and is connected securely to the prosthesis by the threaded stem stud; the inside surface of one wall has a rack gear attached to it and extending the length of the wall; the slide unit comprises a frame with four interconnected parts a front, a back, a first and a second side and a foundation to which the frame parts are connected; the outside dimensions of the frame (length and width) are effectively equal to the inside dimensions of the chamber reduced nominally so that the frame moves smoothly and securely; a pinion (circular) gear engages the rack gear and is mounted on an axle which is secured through the foundation of the slide in the floor of the adjustment base; rotating the pinion gear when it is engaged with the rack gear, moves the rack gear which, in turn, moves the slide unit lengthwise; the slide unit is connected to a ball stud and to the adjustment unit; thus moving the slide unit and attached ball effectively changes the length of the neck and increases pressure on the cup; the magnitude of change in length in indicated directly by reference points on the adjustment base and adjacent edge of the side walls of the slide unit. In an alternative mode by which the length of the neck can be increased or decreased, the adjustment base, slide unit and gears described above are replaced by a screw thread mechanism; a threaded, moveable shaft engages a complimentary threaded bore in the rotating drive block; the shaft extends forward to a pressure transfer plate positioned at the proximal end of the shaft; the transfer plate moves forward or backward in response to rotating the rotating block, thereby rotating the shaft; the ball is connected by a threaded stud to the front edge of the moveable block; the distal end of the shaft is anchored in the base and back wall of the anchor housing; actual distance moved (change in length of the neck) is indicated directly by changes in distance between reference points on the threaded shaft and on the face of the threaded block. In an alternative mode, a worm gear system replaces the rack and pinion gear system, and other parts, as a result, are modified, but the basic concept and scope of the invention remain the same.

### BRIEF DESCRIPTION OF THE FIGURES

FIG. 1 illustrates the basic parts or elements of a hip joint prosthesis

FIG. 2A provides a top view of a schematic illustration of a temporary, modular, adjustable hip joint prosthesis wherein, the neck comprises a gear driven elongation mechanism.

FIG. 2B provides a side view of the prosthesis and gear driven mechanism of FIG. 2A.

FIG. 2C shows details of the gear drive mechanism of FIGS. 2A and 2B.

FIG. 2D provides basic dimensions of the gear drive mechanism of FIG. 2C.

FIG. 2E provides details of the gear lock.

FIG. 3 provides a top view of a screw thread driven elongation mechanism.

FIG. 4 provides alternative, side view of FIG. 3, showing disk structures and orientation.

FIG. 5 illustrates details of the structure and function of the connector bolt.

### EXAMPLES

Introduction

Hip replacement surgery frequently involves only the femoral implant element of the hip prosthesis. The femoral implant may be viewed as comprising four parts or elements: a stem (or peg) that is longitudinally implanted into the femur, a body segment to which the stem is attached, a neck element that, at one end, is attached to the body, and the ball that is attached to the opposite end of the neck element. These elements are obvious in modular prosthetic devices. In some early designs, only the ball was separated from the other elements of the prosthesis, and the base was a generalized area from which the stem and neck extended. In modular devices, the neck is connected to the ball by a first threaded end and to the base by its second end, and similarly, the stem is connected to the base by a threaded end, This modular design is best suited for a temporary implant to allow surgeons to evaluate different size (length) elements to be combined to yield a wide array of sizes to yield the optimum fit of the prosthetic device.

Prosthesis replacement surgery (revision surgery) may present challenges not common to initial prosthetic implant surgery. In spite of significant advancements in replacement technology, not infrequently, during a replacement procedure, placement of the prosthetic device must be modified to ensure achieving optimal results. Commonly this requires refitting the prosthesis, including re-measuring and refitting the device to the amputation site. A frequent example with hip surgery is redetermination of the length of the prosthetic neck to allow optimum positioning of the ball in the acetabulum (cotyloid cup).

A variety of neck lengths may be available for a given base and ball size. Based on all available data, the surgeon selects what appears to be the most suitable combination in a specific case for optimal results. The surgery proceeds and the stem and base positioned and the stem implanted.

When the neck and ball are positioned as initially planned, the ball is not properly aligned in the cup. Frequently, the cause is improper neck length, or at least changing neck length will alleviate the problem. Unfortunately, this type of problem is not easily anticipated. Rarely is the problem clearly observable; most frequently it is determined by the surgeon by “feel” or tension of muscles, ligaments, and tendons supporting the cup: a neck that is too short fails to exert adequate pressure on the cup and generate an appropriate degree of tension on the supporting tissues; too long results in the opposite condition.

With modular prosthetic devices having necks of varying lengths, the common solution has been trial and error, until an acceptable fit is achieved. For any prosthesis, the manufacturers dimensions may be viewed as zero correction in neck length, and adjustments can be made upward (increasing neck length) or downward (decreasing neck length). Practical experience suggests that corrections adjustments are made upward, increasing neck length and that the magnitude of adjustment is greater for increasing neck length than decreasing it. The magnitude of increase or decrease in length is reasonably modest for a given prosthesis. Total scope of correction, by way of example, not of limitation from the manufacturer's base point (0) ranges from −2 to 3 mm to +12 to 15 mm, a range of 14 to 18 mm (about 0.4 inch). Incremental changes are about 2 mm and may practically limited to 1.0-1.5 mm. If a greater magnitude of change is required, replacing the initially selected prosthesis may be required to ensure proper fit of the ball in the cup.

The Basic Femoral, Hip Implant

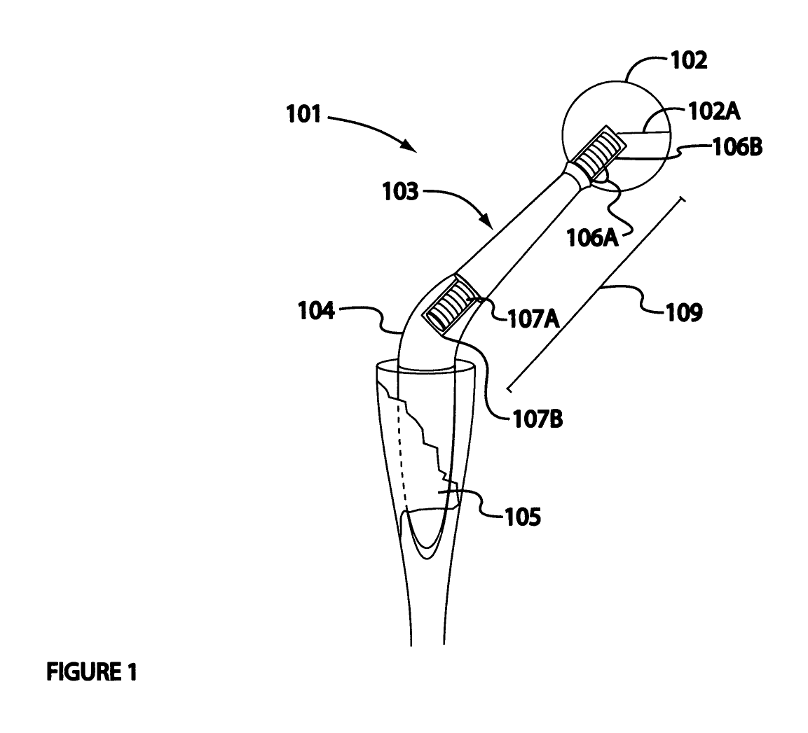

The basic femoral hip implant is well known among those skilled in the art. The following brief summary, including FIG. 1, provides a review of the basic prosthesis that illustrates that illustrates the basic foundation for a longitudinally adjustable, femoral hip prosthesis. The basic femoral hip implant of FIG. 1 clearly identifies the four major elements of the implant 101: the ball 102, the neck 103, the body (or base) 104, and the stem 105.

The neck 103 comprises a first stud 106A and a second stud 107A. The ball 102 comprises a radius 102A and a first threaded receptacle 106B. One skilled in the art understands that the radius of the ball may vary in diameter, by way of example, not limitation, from about 20 mm to over 50 mm (less than 1 in. To over 2 inches), Some preference exists generally for smaller diameter balls (e.g. about 30 mm). Variation in ball size and similar variations do not affect the scope or intent of the claimed invention. The first threaded receptacle 106B is adapted to engage the threaded first stud 106A, thereby securely connecting the neck 103 and ball 102. The stem 105 comprises a second threaded receptacle 107B. The body 104 of the basic femoral implant hip prosthesis comprises a second threaded receptacle 107B adapted to engage the second threaded stud 107A and to securely connect the neck 103 and body 104. Structurally, the stem 105 may be continuous with the body, or the proximal end may be shaped into a third threaded stud, the threaded receptacle for which would be positioned on the body 104. Such a variation has no impact on the scope or intent of the claimed invention.

### Example I

### Neck Length Modification Mechanism

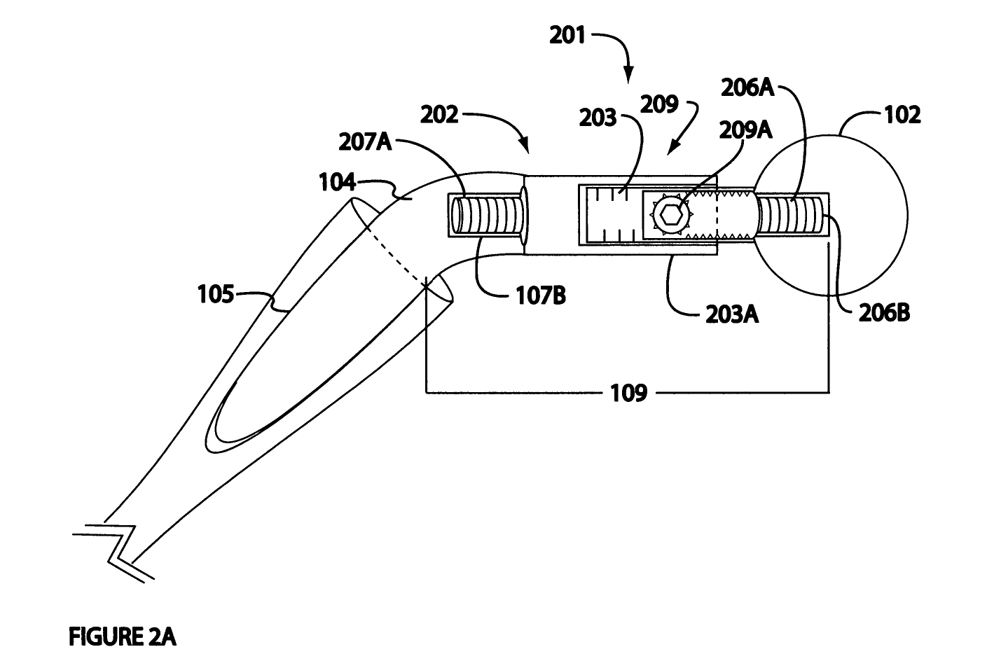

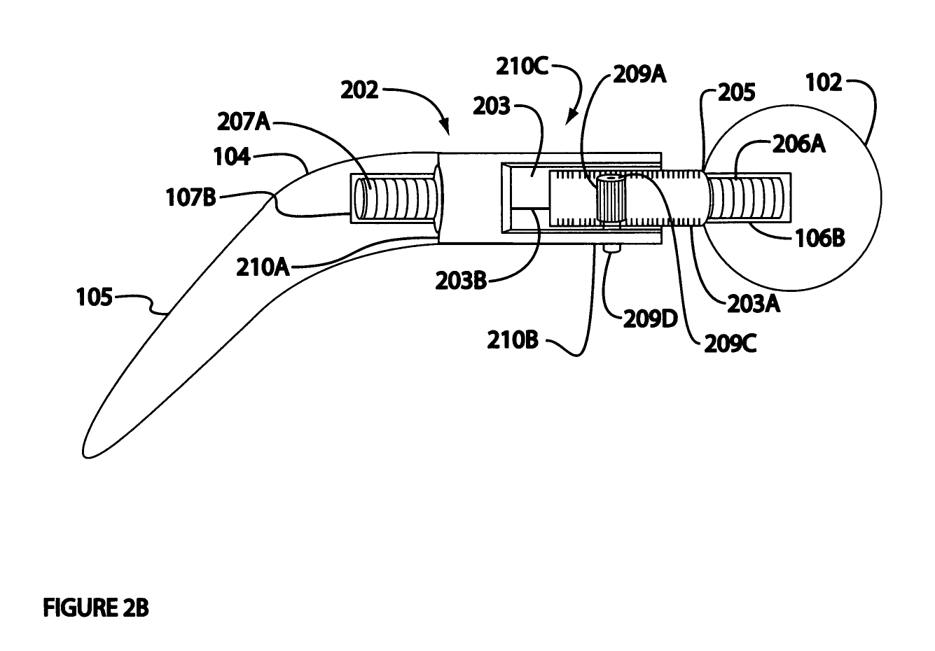

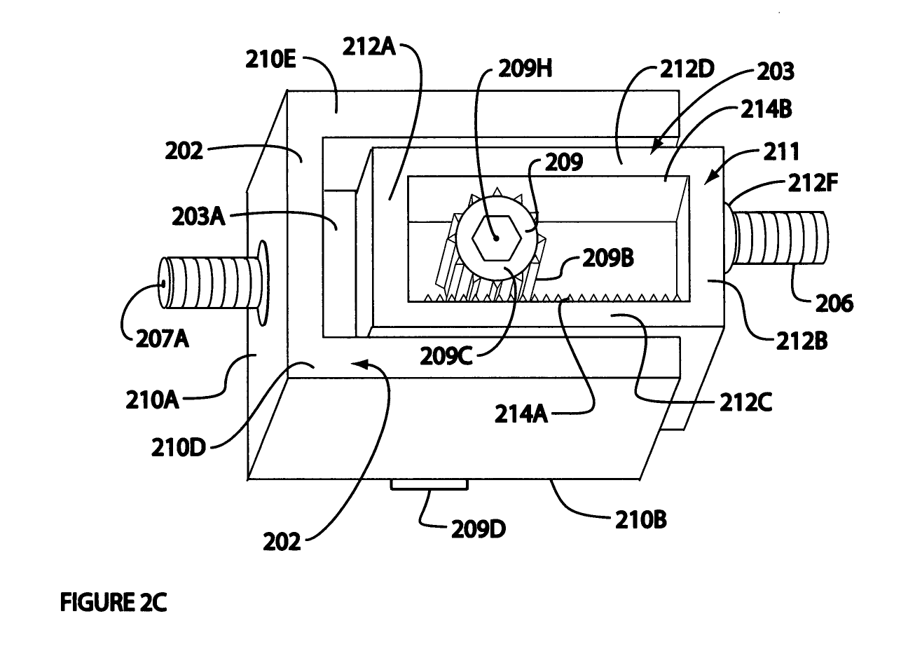

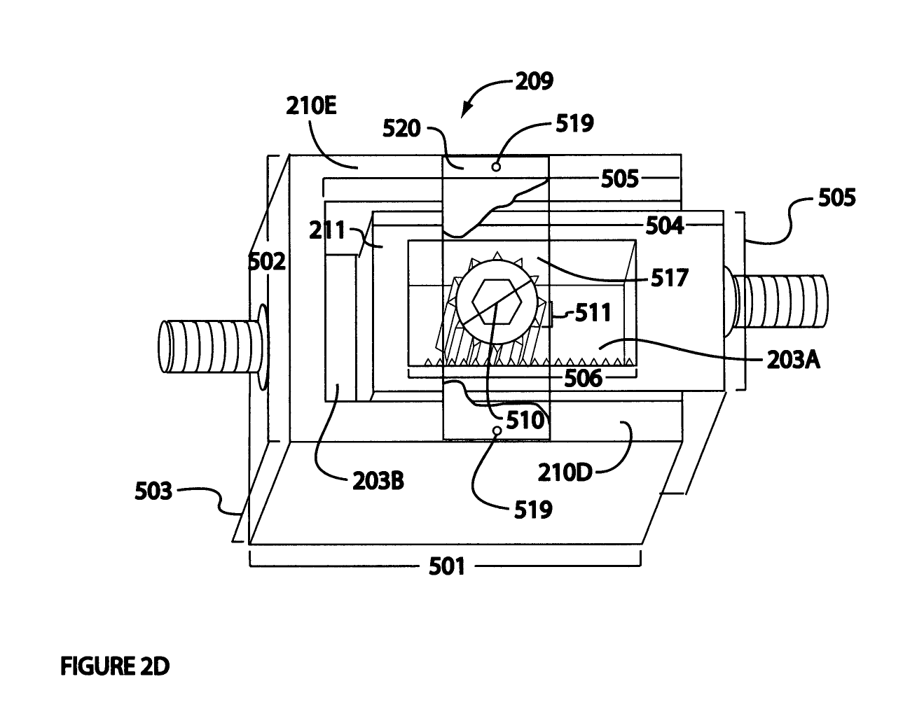

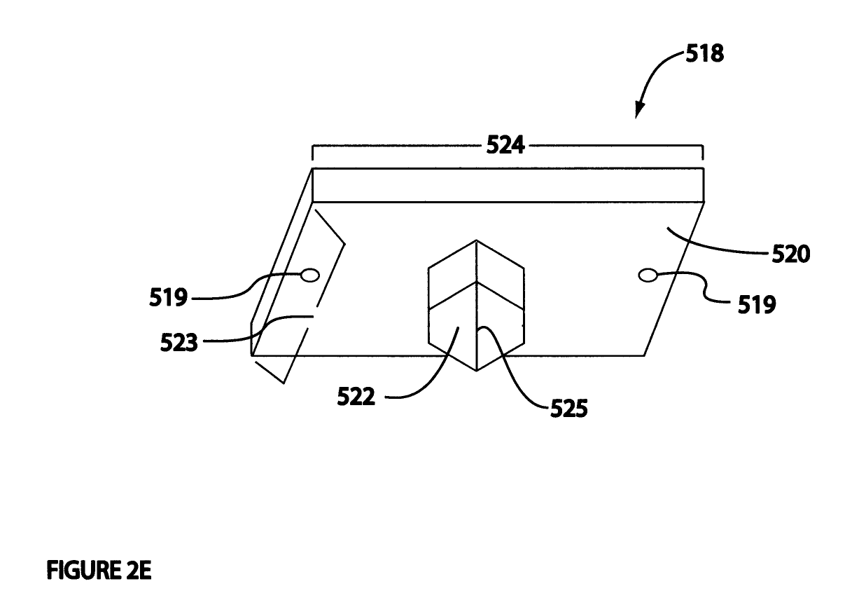

As illustrated in FIGS. 2A and 2B, the neck length modification mechanism 201 comprises a fixed or static component, the adjustment base 202, and a longitudinally moveable component, the slide travel component 203A. The adjustment base 202 further comprises the slide unit chamber, or run 203B. The slide unit chamber 203B comprises a back wall 210A, a first and a second side wall 210D and 210E, respectively, a floor 210B and an open top 210C.

In addition the adjustment base comprises the adjustment base stud 207A. The adjustment base stud 207A is adapted to engage the second receptacle 107B thereby securely connecting the adjustment base to the body, and consequently to the stem.

The slide travel component 203A comprises a box-like structure, the travel gear box 211. The travel gear box 211 is described and limited by a back wall 212A, a front wall 212B, a first side wall 212C and a second side wall 212D, and an open bottom 212E. The threaded slide unit stud 206 is securely fixed to the front wall 212B of the travel gear box and is adapted to functionally engage the ball 102 by receptacle 106B.

In Example 1, FIGS. 2A, B, C, and D, the travel gear system 209 is a variation of a rack and pinion gear system well known to those skilled in the art. The travel gear system 209 (rack and pinion gear system) comprises a comprises a pinion gear 209A that rotates with the axle 209C. The pinion gear 209A is positioned at the center point of the floor 210B. The axle 209C traverses the bottom of the slide unit chamber 210B. The axle 209C is secured in a vertical plane to the floors and rotatably secured in position by an anchor fixture (bushing) 209 secured to the exterior of the slide unit floor 210B. The opposite end of the axle is adapted to receive a tool to rotate the axle. A rack gears 214 is positioned on one of the opposing side walls 212C and 212D and adapted to engage the pinion gear 209A.

The threaded slide unit stud 206 functionally engages the first threaded receptacle 106B, thereby connecting the ball 102 to the static, adjustable base 202. Rotating the axle 209C using a tool (male hex wrench, as shown by way of illustration, not limitation, as one skilled in the art understands). causes the rack gears to move forward (clockwise 209F as illustrated) moves the travel gear box 211 forward thereby moving the ball 102 secured to stud 206. Moving the ball forward increases the pressure of the ball on the hip cup (not illustrated), and the amount of extension required to achieve optimum tension in the opinion of the surgeon can be translated directly into an optimum neck length for the hip prosthesis.

The travel gear system 209 comprises an additional functional element, a gear lock 518. An element that will prevent the axle 209C from inadvertently rotation and allowing a potentially undetected change in the length of the neck. As illustrated in FIG. 2D, only the position of the gear locking body 520 is clearly shown, spanning the width of the adjustable base 202 and secured 519 (bolts, by way of example, not as a restriction) to the top surfaces of the first and second side walls 210D and 210E, respectively.

In FIG. 2D, the middle portion 520A of the gear lock body 520 is cut away 517 so that other elements of the entire device are not masked. Details of the relatively simple gear lock are illustrated in FIG. 2E.

The gear lock 518 comprises a body 520 as described above, and means to connect the body 520 to the first and second side walls 210D and 210E, respectively, of the adjustment base 202. One skilled in the art recognizes that a variety of different means, such as spring clips and similar devices may be used without altering or affecting the scope or intent of the invention. A locking lug 522 is positioned at the center of the inner face of the body. The locking lug 522 may be secured to the body 520 by various means including mechanical (bolts) or physical (welding or the like). The lug 522 has the same configuration as the tool receptacle 209H. As illustrated, this is a hexagon. With the lug 522 in place and the body 520 secured to the side walls 210D and 210E by small bolts or comparable, removable fittings 519. The length 524 of the body 520 is effectively the same as but not less than the distance between the side walls 210D and 210E. The width 523 must be greater than the diameter of the lug 525. With the body properly secured to the side walls the lug, when engaged with tool receptacle 209H, prevents the axle from inadvertent rotation and movement of the ball 102.

FIG. 2D illustrates dimensions in ranges, not specific dimensions. Ranges are given for illustrative purposes, not as specific limitation; in addition, certain dimensions are interdependent, such as wall length and maximum length of the rack gear elements.

By way of illustration, not limitation, the overall length 501 of the mechanism (with the mechanism fully retracted (and excluding stud connectors) varies from 25 to 75 mm, and the overall width 502 varies from 25 to 45 mm, with the height 503 ranging from 20 to 35 mm. The overall length 504 of the slide travel component 203 is effectively equal to the length 505 of the slide chamber unit 203B, and the length of each rack gear 506 is approximately equal to the maximum extension of the mechanism, 26 to 40 mm, by way of example, not limitation.

Each of FIGS. 2A, 2B, 2C, and 2D shows the major components of the neck length modification mechanism 201; however, these individual views should be considered jointly in conjunction with the following text. Index numbers for the same part or feature are constant among the views; however; more specific details are shown in FIGS. 2B, 2C, and 2D than in FIG. 2A. FIG. 2A provides a top view of the basic femoral implant prosthesis 101 in which the ball 102, first receptacle 106B, and radius 102A are the same as in FIG. 1 In addition, the stem 105 body 104, and second threaded receptacle 107B also remain unchanged from FIG. 1. In FIG. 2A, the neck 103 of FIG. 1 is replaced by the neck length modification mechanism 201, comprising two major elements: the adjustment base 202 and the slide component 203. The threaded slide unit stud 206A is adapted to engage and securely connect the ball 102 to the slide component 203. The threaded, adjustment base stud 207A is adapted to engage the second receptacle 107B and securely connect the adjustment base to the body 104 and as a consequence to the stem.

In addition to the adjustment base stud 207A, the adjustment base 202 comprises a square, U-shaped slide unit chamber 203B with a back wall 210A, a first and a second side wall 210D and 210E, respectively, a floor 210B, and an open top 210C. The interior faces of the first and second side walls 212C and 212D, respectively, and the back wall 212E describe and limit the slide unit housing 211.

The slide unit 203 comprises a box-like structure, the travel gear box 211 comprising a rear slide wall 212A, a front slide wall 212B, a first and a second slide wall 212C and 212D, respectively, and an open top and bottom. The first slide wall 212C on its inner face comprises a rack gear element.214. The travel drive 209 is positioned at the center point 209C of the floor 209E of the travel unit 203. The body of the travel guide 209E describes a cylinder rotatably mounted on the floor 210E of the travel unit 203. The vertical face of the body comprises a gear system complimentary to the linear gear 214 and the linear gear is adapted to engage the gear system on the cylinder. When the pinion gear 209A is rotated clockwise (according to FIG. 2B and 2C), the travel element extends forward in response to the gear engagement between the complimentary gear system of the pinion gear and the rack gear. This movement is transferred to the ball and to the cup as increased pressure on the cup that tightens the ball in the cup. When rotation of the cylinder is reversed, the slide travel element 203 retracts and the ball exerts less pressure on the cup and is tension holding the ball in place in the cup is reduced. The maximum extension/retraction is limited by the length of the of the rack gears. Effectively, to accommodate increase and/or decrease in length, the length of the slide unit must be twice the length of the rack gear 214. Thus the length of the travel space 208 varies with the rotation of the pinion gear 209A.

The travel gear system 209 comprises a pinion gear 209A and an axle 209C with which the pinion gear rotates; the upper end of the axle comprises a tool receptacle 209H that is adapted to engage a hand tool that can rotates the axle 209C. The axle is secured to the floor 210B of the adjustable base 202 that includes a bushing or comparable anchor/rotation fixture point 209D positioned to extend to the exterior of the floor 210B of the adjustable base 202. The pinion gear is positioned at the center point of the travel gear box 211 such that it is adapted to engage the rack gear 214.

FIG. 2C illustrates the neck length modification mechanism extended as indicated by the length of the travel 208A and the position the pinion gear 209A relative to the rack gears 214A and 214B compared to the length of travel 208B in FIG. 2B.

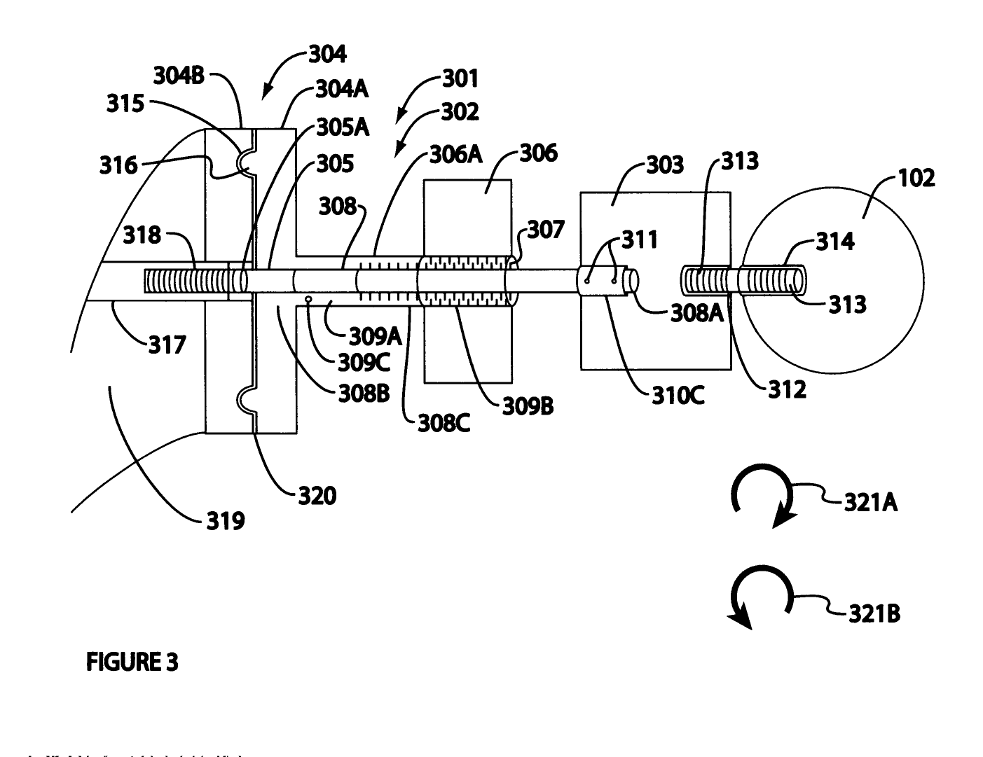

Referring to FIG. 2D, the length 504 of the slide unit housing 211 is nominally twice the length 305 of the rack gear 214. The rack gear effectively defines the maximum length of travel of the slide unit. Hence extension of the neck and the movement of the ball in the cup. The length of the neck 103 limits the maximum length (overall) of the modification and the diameter of the neck suggests the maximum width of the modified neck as illustrated in FIG. 3.

The overall length 504 of the slide unit 203 extends from the back 210A of the adjustment base 202 to the front wall 203A of the slide unit 203; This distance varies by a length up to the length of up to the travel distance 208 (see also 208A FIG. 2A and 208B FIG. 2B) as a direct function of the extension/retraction of the slide unit travel in response to the rotation of the pinion gear 209 and its functional contact with the rack gears 214.

The maximum travel, by way of example, not as a strict limitation, varies from about 20 mm and 40 mm, maximum with an average of about 25 mm (1 inch).

The relation between the rotation of the pinion gear and distance traveled by the rack gear (extension or retraction distance) is a direct function of the diameter of the pinion gear 209A and number of teeth per unit of length as calculated by the distance between teeth and resultant spacing per unit of length of the rack rears.

### Example II

### Alternative Neck Length Modification Mechanism

FIG. 3 provides a schematic, side view of the neck length modification mechanism 301 of second example. The purpose of the alternative mechanism in Example II is effectively the same as that for the in Example I: to provide the surgeon with a temporary hip prosthetic device the length of the neck of which can be modified without removal of the entire prosthesis so that the optimum neck length may be more accurately and more rapidly determined during the course of the implant procedure. Although the both functional and structural similarities exist between the mechanism of Example I and of Example II, the two mechanisms differ to a degree such that, except for the ball 102. Parts and functions in this example are assigned new names and index numbers.

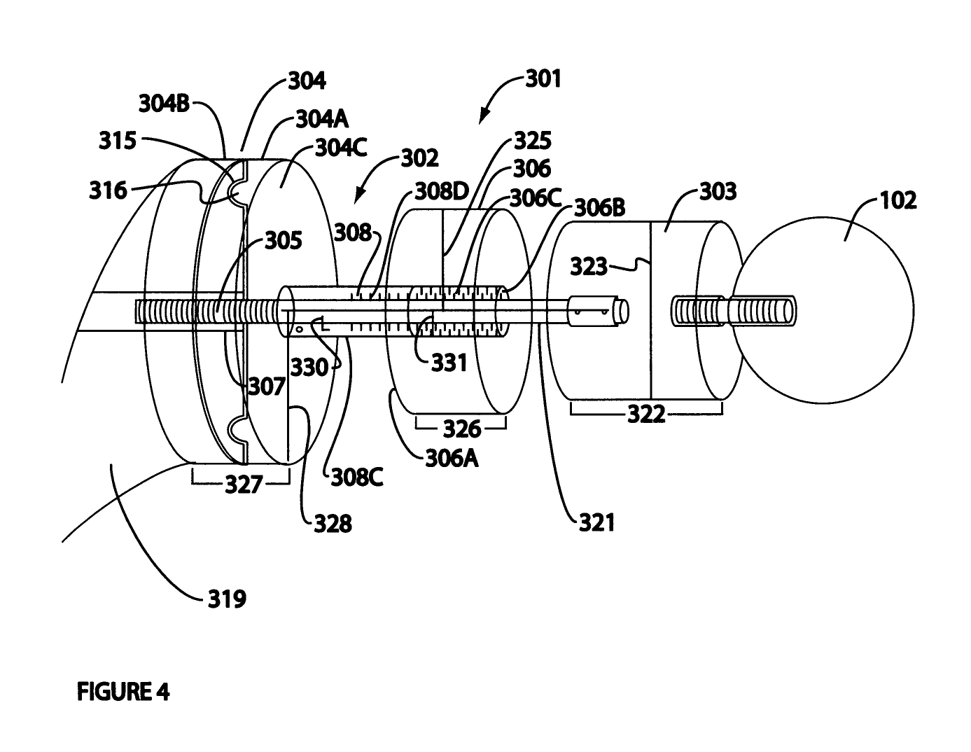

The second neck length modifying mechanism 301 comprises three main elements: a rotatable, adjustable base 302, a pressure disk 303, and the male, threaded drive axle 308. Note, the several disk housings in FIG. 3 are in fact disk-shaped as shown in FIG. 4. The use of blocks simplified illustrating certain relationships without in any way altering the scope or intentions of the claimed invention.

The rotatable, adjustment base 302 comprises the largest element of the mechanism 301 and comprises several parts.

Starting at the prosthesis base 319 and moving to the ball 102 at the opposite end, the rotatable, adjustment base 302 comprises the connector disk 304; the connector disk 304 comprises two units the rotatable disk unit 304A and the static disk 304B. The rotatable disk 304A and static disk 304B are functionally connected by a disk connector bolt 305 and the smooth, proximal end of the disk connector bolt rotatably engages the smooth, proximal end segment of the disk connector receptacle, and further wherein the disk connector bolt secures the rotatable disk to the static disk.

The rotatable disk 304A and the static disk 304B are connected by the disk connector bolt 305. The disk connector bolt 305 comprises a threaded distal segment 305B, a smooth proximal segment 305A, and a head 305C. The disk connector bolt bore 307 traverses both the rotatable disk unit 304A and the static disk unit 304B of the connector disk 304. The threaded distal end 307B of the disk connector bolt bore 307 functionally engages the threaded, distal end of the disk connector bolt 305B

Said connector disk 304 is physically and functionally connected to said drive disk 306 by a bridge 306A, and said bridge 306A is traversed by said connector bolt chase 307; said connector bolt chase functionally houses said drive axle 308

The static disk 304B is secured functionally against the rotatable disk 304A by the recessed head 305C of the disk connector bolt 305 exerting pressure on the rotatable disk 305A. The opposing faces of the rotatable disk 304A and the static disk 304B are separated by a thin washer 317. In addition, a shallow groove 315 circumscribes the perimeter of the static disk 304B and a complimentary rail 316 circumscribes the opposing face rotatable disk 304A. The rail 316 engages the groove 315 as the disk connector bore is tightened. The groove 315 and rail 316 are position, for example, not limitation, about 1.5 to 2.5 mm from the outer perimeter of the static and rotatable disks 304B and 304A, respectively. The groove 315 is smooth and U-shaped, and, by way of example, not limitation, about 1 mm wide and 1.5 mm deep. The rotatable disk 304A rotates around the smooth, proximal end 305A of the disk connector bolt 305. The rotatable disk 304A effectively rides on the rail in the groove to maintain precise alignment of the static and rotatable disks 304B and 304A, respectively. The disk connector bolt is tightened adequate tight to prevent free rotation of the rotating disk 304A and not so tight as to prevent rotation of the rotatable, adjustable base 302.

The rotatable adjustable base 302 further comprises an axle housing 308C that extends from the first face 306A of the drive disk 306 to the second face 304C of the rotating disk 304A. The axle housing 308C is the physical link between the rotatable disk 304A of the connector disk 304 and the drive disk 306. The inside diameter of the axle housing 308C (from radius line 331) is nominally equal to the outside diameter of the threaded, drive axle 308. With the exception of the threaded bore drive 306B, that segment of the axle housing 308C entirely within the bore drive 306C the interior walls of the axle housing 308C are not threaded and these walls describe and limit the open core (lumen) of the axle housing 308C; however, the length of the axle housing, indicated by center line 329 (20 to 40 mm, by way of example, not limitation) is adequate to accommodate the axle 308 as it travels backwards (from the ball 102) and forward (towards the ball 102) through the through the threaded drive bore 306B in response to rotation of drive disk 306, that in fact rotates the entire rotatable adjustment base 302, except for the static disk 304B.

Finally, the rotatable adjustable base 302 comprises a drive disk 306. The drive disk 306 comprises the threaded drive bore 306B. The bore threads 306C are complimentary to and functionally engage the axle threads 308D. The inside radius 330 of the threaded, drive bore 306B is the same as the inside radius of the axle housing 308C. The drive disk is structurally contiguous with the axle housing and the axle housing is contiguous with the second face 304C of the static disk, and with the disk connector bolt securely holding the static 304B and rotatable disk 394A, as a single part, the rotatable adjustable disk becomes a single functional unit. The bore threads 306C engage the axle threads 308D; the bore threads 306C rotate as the drive disk 306 rotates in response to an external, manually applied force.

The first end 308A of the drive axle 308 is physically secured by at least one pin 311 (or comparable fitting well known to those skilled in the art), to the first pressure disk receptacle 310C, and the second end 308B of the drive axle 308 is free in the axle housing 308C. Thus, when the drive disk 306 is rotated clockwise 321A, because the components of the rotatable adjustment base 302 (drive disk 306; axle housing 308C, and connector disk 304) are interconnected and ultimately secured through the prosthesis base 319, rotation moves the drive axle 308 forward, thereby moving the ball 102 forward as a result of forward pressure transmitted through movement of the axle to the pressure disk 303. Because the drive axle 308 is attached only to the pressure disk 303 and because the static dish 304B and rotatable disk 304A are connected by the disk connector bolt 305 that allows only the rotator disk to turn and the static disk secures the static disk 304 and in effect the entire rotatable adjustment base to the prosthesis base and through it to the stem which is implanted in the femur, rotating the drive disk affects only the axle in terms of movement, and moving the ball forward reflects effective lengthening the neck from its original position.

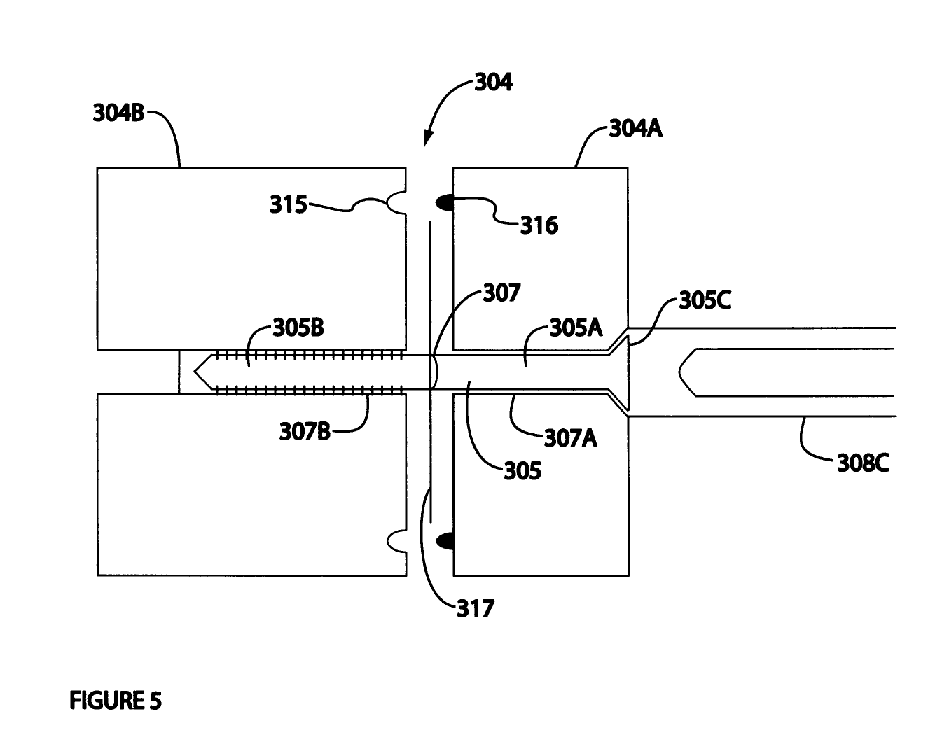

FIG. 4 provides a schematic illustration of the disk shapes of the pressure disk 303, with its length 322 and radius 323, of the drive disk 306 with its length 326 and radius 325 and of the elements of the connector disk 304 with disk overall length 327 and radius 328. The radius of the rotatable disk 304A and of the static disk 304B are equal and the length of each effectively one-half of the length 327 of the connector disk. In addition the length of the threaded, drive axle 308 and radius 330 are shown. As shown in FIG. 3, said rotatable adjustable base 302 is connected to the stem 319 by a separate connecting lug 318 positioned in the receptacle 317; see also FIG. 5.

The specific dimensions of the second neck length modification mechanism, like those of the first example, are mutually interdependent. The specific length of the second neck length modification mechanism varies with the design and dimensions of the permanent prosthesis to be implanted. The overall length 329, with the neck length modification fully retracted will not exceed the length of the permanent prosthetic device selected by the surgeon. The length 329 will be the sum of the lengths of the connector disk 327, the drive disk 306, the pressure disk 303, and the axle housing 308C that connects the drive disk to the face of the rotatable disk 304A and the pressure disk 303. The lengths of the various disks vary, some with greater option than others. By way of example, not limitation, the length on the static disk must represent adequate thickness be adequate to secure the threaded end 305B of the disk connector bolt 305 and on the same centerline for a receptacle to secure the static disk 304B to the prosthesis base 319, and the length of the rotatable disk 304A must only provide support to allow the rotatable disk to turn on the smooth end 305A and for the bolt head 305C to be recessed in the face of the bolt; thus the length could be equal so long as it is based on the static disk (a minimum of 16-17 mm) and the rotatable disk could be reduced to 11-12 mm). The diameter of the two disks 304A and 394B are equal and generally are approximately equal to, or slightly less than the diameter of the corresponding face of the prosthesis base 319. This may vary from less than 25 mm to 50 mm. The length 326 of the drive disk 306 is a direct function of the thread spacing on the axle and drive bore 306B. One skilled in the art recognizes that this spacing determines the extension of the axle with each rotation of the drive disk 306. Diameter of the drive disk is not critical, but in practice it would be less than the diameter of the connector disk 304. Length and diameter of the pressure disk 303 are comparable to the drive disk considering that the drive axle 308 and ball stud 102A must be aligned on the same center line.

The several parts, units, and elements described in Example I and in Example II are specific for each unique example. One skilled in the art recognizes that these parts, units, and elements may be combined and/or combined into additional examples that address the same purpose and functions as Example I and Example II. The application recognizes and claims them as part of the invention. As a result, the appended claims which are based on Examples I and II should be accorded the broadest reasonable interpretation and application.

We claim:

1. A prosthetic hip, neck-length modification mechanism comprising, in a functional linear arrangement of, a base element, a gear system, wherein said gear system, and a travel element and, wherein said base element is secured to the stem of the prosthetic hip and wherein, said prosthetic hip, neck-length modification mechanism is a temporary part of said prosthetic hip; and wherein, said base element is directly connected to said gear system, and wherein said gear system comprises a first gear and a second gear wherein the axle element of said first gear rotates around as fixed point and is adapted to engage the second mobile gear, and wherein structurally, wherein said second mobile gear moves in a linear path connecting said gear second gear with said travel unit and wherein said travel element is in direct contact with an element of the ball element of the hip prosthesis and wherein said ball moves directly in response to the movement of said travel gear movement and wherein rotation of said static gear is the initial force that is transferred to said second gear the ultimate modification in the length of the prosthetic neck

2. The prosthetic, hip, neck-length modification mechanism of claim 1, wherein said prosthetic hip, neck-length modification mechanism comprising, functionally and structurally, in a linear arrange a static adjustment base, a slide travel compartment, a moveable travel gear box, wherein said second gear comprises a rack and pinion gear system, wherein said adjustment base further comprises an adjustable base stud that physically connects said adjustment base to the implantable stem of a hip prosthetic device, and further, wherein the interior surface of the outermost first back end, first side, second side, and bottom walls of said static base define and limit said travel compartment, and wherein the members of the of innermost walls—back wall, front wall, and first and second side walls describe and limit the travel gear box, and further, wherein a threaded slide unit stud is securely attached to said innermost, first front wall of said travel gear box; and further, wherein, said travel gear box comprises the gear system wherein said travel gear system comprises an axle, a pinion gear, and a rack gear, and wherein, said pinion gear is mounted on said axle and positioned on a center point on said floor of said adjustable base, and said rack gear is positioned on and secured to an inner opposing wall of said travel gear box such that said rack gear functionally engages said pinion gear; and further, wherein, said axle traverses said floor and is secured by a bushing such that the said pinion gear revolves with said axle; and wherein the end of said axle opposite said bushing is adapted to engage a tool designed to rotate said axle and pinion gear, and when said pinion gear and rack gear are functionally engaged, said gear travel box moves linearly, thereby changing the length of said neck to the same degree and in the same direction.

3. The prosthetic, hip, neck-length modification mechanism of claim 2 wherein the adjustable base includes a gear lock

4. The prosthetic, hip, neck-length modification mechanism of claim 2 wherein the adjustable base moveable travel gear and gear system are manufactured from medical grade stainless steel and related alloys, including titanium.

5. The prosthetic hip, neck-length modification of mechanism in claim 2 wherein the adjustable base moveable travel gear and gear system are made of appropriate, sterilizable materials, other than stainless steel and alloys including titanium.

6. The hip prosthetic, neck-length modification mechanism of claim 1, wherein said prosthetic, hip, neck-length modification mechanism further comprises a rotational adjustment base, a pressure disk, a rotating drive disk, wherein said rotating drive disk comprises a threaded drive axle, and further, wherein, said threaded drive axle is physically and functionally connected to said pressure disk; and further, wherein said rotational base comprises a connector disk, and further, wherein said connector disk comprises a static disk and a rotatable disk, and wherein said static disk is physically connected to the prosthesis base, and further wherein said static disk and said rotatable disk are connected by the disk connector bolt; and wherein, said connector bolt is connected to said drive disk by said connector bolt bore, and further, wherein said connector bolt functionally houses the said axle; and wherein said drive axle extends from said static disk wherein said static disk is secured by at least one axle pin; and further, wherein, said static disk is secured functionally against said rotatable disk by tightening the recessed head of said disk connector bolt, thereby exerting pressure on said rotatable disk; moreover, a shallow groove circumscribes the perimeter of said static disk and a complimentary rail circumscribes the perimeter of the opposing face of said rotational disk, wherein said rail engages said groove as said disk connector bolt is tightened by tightening the recessed head of said disk connector bolt against the recess in the second face of said rotating disk; wherein said disk connector bolt is tightened adequately to prevent free rotation of said rotating disk and not so tight as to prevent rotation of said rotatable, adjustable base, and finally, wherein the gear system of the hip, prosthetic, neck-length modification mechanism comprises a worm gear system, wherein the worm gear element of said worm gear system comprises the threaded portion of said drive axle and wherein the threaded distal end of the disk connector bolt bore comprises the static element of said the worm gear system, and said threaded portion of said drive axle comprises the mobile element of said worm gear system.

7. The prosthetic hip, hip, neck-length modification mechanism of claim 6, wherein said mechanism is manufactured from medical grade stainless steel and related alloys, including titanium.

8. The prosthetic, hip, neck-length modification mechanism of claim 6 wherein said mechanism is made of any appropriate, sterilizable material, other than stainless steel and related alloys including titanium.
